(19) United States
(12) Patent Application Publication
(10) Pub. No.: US 2007/0244080 A1
(43) Pub. Date: Oct. 18, 2007

(54) Method for Treatment of Inflammatory Disorders Using Triptolide Compounds

(75) Inventors: John M. Fidler, Oakland, CA (US); John H. Musser, San Carlos, CA (US)

Correspondence Address: PERKINS COIE LLP, P.O. BOX 2168, MENLO PARK, CA, 94026, US

(73) Assignee: PHARMAGENESIS, INC., Palo Alto, CA (US)

(21) Appl. No.: 11/629,747

(22) PCT Filed: Jun. 23, 2005
(86) PCT No.: PCT/US05/22247

§ 371 Date: Jul. 5, 2007

Related U.S. Application Data
(60) Provisional application No. 60/583,295, filed on Jun. 25, 2004.

(51) Int. Cl. A61K 31/56; A61P 29/00

(52) U.S. Cl. 514/172

(57) ABSTRACT

Inflammatory disorders, including obliterative airway disease, renal fibrosis, diabetic nephropathy, and liver fibrosis are treated with immunosuppressive triptolide compounds, in particular triptolide compounds effective to inhibit TGF-β production in a patient afflicted with such a disorder.

### FIELD OF THE INVENTION

The invention is directed to treatment of inflammatory disorders, including obliterative airway disease, renal fibrosis, diabetic nephropathy, and liver fibrosis, and in particular to use of triptolide compounds to inhibit TGF-β production in a patient afflicted with such a disorder.

### REFERENCES

Chen, B. J., Chen, Y., Cui, X., Fidler, J. M., and Chao, N. J. Mechanisms of tolerance induced by PG490-88 in a bone marrow transplantation model. Transplantation 73:115 (2002).

Chen, B. J., Liu, C., Cui, X., Fidler, J. M. and Chao, N. J. Prevention of graft-versus-host disease by a novel irnrnunosuppressant, PG490-88, through inhibition of alloreactive T cell expansion. Transplantation 70:1442 (2000).

Fidler, J. M., Ku, G. Y., Piazza, D., Xu, R., Jin, R., and Chen, Z. Immunosuppressive activity of the Chinese medicinal plant Tripterygium wilfordii. III. Suppression of graft-versus-host disease in murine allogeneic bone marrow transplantation by the PG27 extract. Transplantation 74:445 (2002).

Gabbiani, G. The myofibroblast in wound healing and fibrocontractive diseases. J. Pathol. 200:500-3 (2003).

Goto, Y., N. Manabe, K. Uchio-Yamada, M. Yamaguchi-Yarnada, N. Inoue, Y. Yamamoto, A. Ogura, N. Nagano and H. Miyamoto. Augmented cytoplasmic Smad4 induces acceleration of TGF-β1 signaling in renal tubulointerstitial cells of hereditary nephrotic ICGN mice with chronic renal fibrosis; possible role for myofibroblastic differentiation. Cell Tissue Res. 315:209-21 (2004).

Gross, T. J., G. W. Hunninghake. Idiopathic pulmonary fibrosis. N. Engl. J. Med. 345:517-525 (2001).

Jerums, G., S. Panagiotopoulos, J. Forbes, T. Osicka and M. Cooper. Evolving concepts in advanced glycation, diabetic nephropathy, and diabetic vascular disease. Arch. Biochein. Biophys. 419:55-62 (2003).

Kershenobich-Stalnikowitz, D. and A. B. Weissbrod. Liver fibrosis and inflammation. A review. Ann. Hepatol. 2:159-63 (2003).

Mason, R. J., M. I. Schwarz, G. W. Hunninghake and R. A. Musson. NHLBI workshop summary: pharmacological therapy for idiopathic pulmonary fibrosis: past, present and future. Am. J. Respir. Crit. Care Med. 160:1771-1777 (1999).

Qiu, D. and Kao, P. N. Immunosuppressive and anti-inflammatory mechanisms of triptolide, the principal active diterpenoid from the Chinese medicinal herb Tripterygium wilfordii Hook. f. Drugs R. D. 4:1 (2003).

Qiu, D., Zhao, G., Aoki, Y., Shi, L., Uyei, A., Nazarian, S., Ng, J. C. and Kao, P. N. Immunosuppressant PG490 (Triptolide) inhibits T-cell interleukin-2 expression at the level of purine-box/Nuclear factor of activated T-cells and NF-kappaB transcriptional activation. J. Biol. Chem. 274:13443 (1999).

Schlesinger C, Meyer C A, Veeraraghavan S, Koss M N. Constrictive (obliterative) bronchiolitis: diagnosis, etiology, and a critical review of the literature. Ann Diagn Pathol. 2:321-34 (1998a).

Schlesinger, C, Veeraraghavan S, Koss M N. Constructive (obliterative) bronchiolitis. Curr Opin Pulm Med. 4:288-93 (1998b).

Selman M., T. E. King and A. Pardo. Idiopathic pulmonary fibrosis: prevailing and evolving hypotheses about its pathogenesis and implications for therapy. Ann. Intern. Med. 134:136-151 (2001).

Wang, J., Xu, R., Jin, R., Chen, Z. and Fidler, J. M. Immunosuppressive activity of the Chinese medicinal plant Tripterygium wilfordii. I. Prolongation of rat cardiac and renal allograft survival by the PG27 extract and immunosuppressive synergy in combination therapy with cyclosporine. Transplantation 70:447 (2000).

Yamagishi, S., Y. Inagaki, T. Okamoto, S. Amano, K. Koga and M. Takeuchi. Advanced glycation end products inhibit de novo protein synthesis and induce TGF-β overexpression in proximal tubular cells. Kidney Int. 63:464-73 (2003).

### BACKGROUND OF THE INVENTION

Obliterative airway disease (OAD) is characterized by airflow obstruction and pathological findings showing constrictive bronchiolitis (bronchiolitis obliterans). OAD may result from chronic rejection of lung transplants, the main factor that limits long-term transplant survival to approximately 50% at 5 years despite aggressive immunosuppressive therapy. Other causes of OAD include exposure to toxic fumes, infection, and bone marrow transplantation (Schlesinger et al., 1998a; 1998b). Patients with the constrictive pattern of bronchiolitis often do not respond to corticosteroid therapy, and prognosis is poor.

Tubulointerstitial fibrosis (renal fibrosis) contributes to the progression of many forms of glomerular disease and to end-stage renal failure. In the hereditary mouse model of nephrotic syndrome with chronic renal tubulointerstitial fibrosis, tubulointerstitial cells differentiate to myofibroblastic cells that are positive for alpha-smooth muscle actin (Goto et al., 2004). Renal fibrosis is the final common pathway for many kidney lesions that lead to chronic progressive organ failure.

Hepatic (liver) fibrosis is a wound healing process characterized by accumulation of extracellular matrix proteins (ECM), especially collagen, as well as an increase in other ECM constituents such as proteoglycans, fibronectin and laminin in response to liver injury (Kershenobich-Stalnikowitz et al., 2003). Proinflammatory and profibrotic cytokine production is a major contributing factor to the accumulation of extracellular collagen in liver fibrosis.

Proinflammatory and profibrotic cytokine production is also a major contributing factor to the accumulation of extracellular collagen in diabetic nephropathy, which appears to result from the actions of cytokines and growth factors.

### SUMMARY OF THE INVENTION

The invention provides, in one aspect, a method of inhibiting cytokine production, particularly TGF-β production, in a patient infected with a disorder selected from obliterative airway disease, renal fibrosis, diabetic nephropathy, and liver fibrosis, thereby reducing symptoms of the disease, comprising administering to such a patient an immunosuppressive triptolide compound. The triptolide compound may be triptolide, a prodrug of triptolide, an immunosuppressive derivative of triptolide, or a prodrug thereof In one embodiment, the triptolide compound is an immunosuppressive derivative of triptolide or a prodrug thereof. Such triptolide compounds are described further below.

The triptolide compound may be employed in combination with an additional therapeutic agent selected from an antiviral agent, an antiinflammatory agent, such as a corticosteroid, an additional immunosuppressive agent, and an immune potentiator. Such agents are also described further below.

### DETAILED DESCRIPTION OF THE INVENTION

I. Triptolide Compounds

In accordance with the present invention, triptolide, triptolide derivatives and triptolide prodrugs (referred to collectively as triptolide compounds) are effective to inhibit TGF-β production and are useful in the treatment of disorders such as obliterative airway disease, renal fibrosis, diabetic nephropathy, and liver fibrosis.

The compound triptolide, a diterpene triepoxide isolated from the Chinese medicinal plant Tripterygium wilfordii, has potent immunosuppressive and antiinflammatory properties and reduces T lymphocyte proliferation and recruitment (Qui et al., 1999).

Triptolide suppresses in vitro production of proinflammatory cytokines such as IFN-γ, TNF-α, IL-1β and IL-6, as shown in Table 1. To obtain the data, Jurkat cells were stimulated for 8 hr by PMA and ionomycin. Human peripheral blood mononuclear cells (PBMC) from a single donor were incubated for 24 hr with PHA. At the end of the culture period, each supernatant was harvested, and the cytokine content was assayed by ELISA.

TABLE 1
Suppression of cytokine production by triptolide

| Cells and stimulus | Cytokine | IC₅₀ (ng/ml) |
|---|---|---|
| PMA/ionomycin-induced | IL-2 | 1.3 |
| Jurkat cells | | |
| PHA-induced PBMC | IL-1β | 0.45 |
| | IL-2 | 0.38 |
| | IL-6 | 1.5 |
| | TNF-α | 0.35 |
| | IFN-γ | 0.52 |

Triptolide suppresses the production of cytokines in a variety of in vitro systems. For example, triptolide inhibits early cytokine gene expression in Jurkat T cells, effectively suppressing T lymphocyte activation (Qui et al., 1999). Triptolide inhibits production of IL-2 in activated human peripheral blood mononuclear cells (PBMC) and in activated Jurkat cells (Table 1; see Qui et al., 1999, 2003). The secretion of the proinflammatory cytokines IFN-γ, TNF-α, IL-1β and IL-6 by PHA-activated human PBMC is also suppressed by triptolide (Table 1). Triptolide inhibits the expression of several cytokine genes in activated Jurkat cells, including IL-2, IL-3, IL-6, IL-8, IL-13, TNF-α, TGF-β, MIP-1α, MIP-1β, GM-CSF and RANTES (Qui et al., 2003). In addition to its effects on immune cells, triptolide suppresses IL-8 expression by bronchial epithelial cells, inhibiting both IL-8 mRNA and IL-8 protein expression (Qui et al., 1999).

Triptolide derivatives and prodrugs which can be used in various embodiments of the invention include those described in several co-owned US patents, including U.S. Pat. Nos. 5,663,335, 6,150,539, 6,458,537, 5,962,516, and 6,569,893, and in co-owned PCT Pubn. No. WO 2003/101951, each of which is hereby incorporated by reference in its entirety. These derivatives and prodrugs can be prepared from triptolide by one of ordinary skill in the art, according to standard methods of organic synthesis, as described therein. Several examples are provided below.

For the purposes of the current disclosure, the following numbering scheme is used for triptolide compounds:

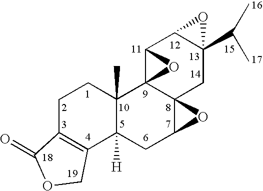

An exemplary triptolide prodrug, triptolide 14-succinate (designated PG490-88; see U.S. Pat. No. 5,663,335), is converted in vivo to triptolide by the action of esterases in plasma. The compound has shown in vitro activity in suppression of IL-2 production after incubation in plasma, and has shown efficacy in several animal models of immunosuppression (see e.g. Chen et al., 2000; Wang et al., 2000; Chen et al., 2002; Fidler et al., 2002).

Further exemplary triptolide derivatives and prodrugs which can be used in various embodiments of the invention include 14-methyl triptolide (designated PG670; see US Application Pubn. No. 20040152767), triptolide 14-tert-butyl carbonate (designated PG695) and triptolide 14-ethyl carbamate (designated PG666) (see US Application Pubn. No. 20040235943 and corresponding PCT Pubn. No. WO 03/101951), 14-deoxy-14α-fluoro triptolide (designated PG763; see US Application Pubn. No. 20040198808), triptolide 14-(α-dimethylamino)acetate (designated PG702; see U.S. Pat. No. 5,663,335), 5-α-hydroxy triptolide (designated PG701; see U.S. application Ser. No. 60/532,702), 14-acetyl-5,6-didehydro triptolide (designated PG746; see U.S. application Ser. No. 60/532,702), 19-methyl triptolide (designated PG795; see U.S. application Ser. No. 60/549,769), and 18-deoxo-19-dehydro-18-benzoyloxy-19-benzoyl triptolide (designated PG796; see U.S. application Ser. No. 60/549,769).

Each of these compounds has demonstrated significant cytokine inhibiting activity, as shown in the above-referenced patents and applications. For example, PG796 (18-deoxo-19-dehydro-18-benzoyloxy-19-benzoyl triptolide) showed a higher level of activity in a standard IL-2 inhibition assay than the triptolide prodrug, triptolide 14-succinate. Both 5α-hydroxy triptolide (designated PG701) and 14-acetyl-5,6-didehydro triptolide (designated PG746) inhibited IL-2 production in Jurkat cells in a dose-dependent manner at concentrations of about 10 nM or greater (the latter after incubation for 16 hours with human serum, which presumably removes the acetyl group). The activity of PG763 (14-deoxy-14α-fluoro triptolide) in assays evaluating cytotoxicity and IL-2 inhibition was nearly equivalent to that of native triptolide. Each of 19-methyl triptolide (designated PG795), triptolide 14-ethyl carbamate (designated PG666), and 14-methyl triptolide (designated PG670) showed equal IL-2 inhibitory activity to triptolide at about 10-30 times the active concentration of triptolide.

Convenient methods of preparation of these and related compounds are described in the above-referenced patents and applications, and several exemplary procedures are reproduced below in the Examples. Each of these US applications and patents is hereby incorporated by reference in its entirety.

Any of the above triptolide compounds having an ionizable group at physiological pH may be provided as a pharmaceutically acceptable salt. This term encompasses, for example, carboxylate salts having organic and inorganic cations, such as alkali and alkaline earth metal cations (for example, lithium, sodium, potassium, magnesium, barium and calcium); ammonium; or organic cations, for example, dibenzylammonium, benzylammonium, 2-hydroxyethylammonium, bis(2-hydroxyethyl)ammonium, phenylethylbenzylammonium, dibenzylethylenediammonium, and the like. Other suitable cations include the protonated forms of basic amino acids such as glycine, ornithine, histidine, phenylglycine, lysine, and arginine.

Many of these triptolide compounds act as prodrugs, by converting in vivo to triptolide, as observed for PG490-88, above. Such compounds are expected to convert to triptolide in vivo by known mechanisms, such as hydrolysis of an ester (organic or inorganic), carbonate or carbamate to an alcohol, or ring opening or ring closure from or to an epoxide or lactone. Such conversion is readily evaluated in vitro by incubating in blood serum, as described in the Examples below. Conversion times may vary according to the steric and electronic characteristics of the converting moiety. Such prodrug compounds are typically designed with such conversion in mind. These include, of those noted above, triptolide 14-succinate, triptolide 14-ethyl carbamate, triptolide 14-t-butyl carbonate, and triptolide 14-(α-dimethylamino) acetate.

Other triptolide compounds, such as 14-deoxy-14α-fluoro triptolide, 14-methyl triptolide, 5-α-hydroxy triptolide, 14-acetyl-5,6-didehydro triptolide, 19-methyl triptolide, and 18-deoxo-19-dehydro-18-benzoyloxy-19-benzoyl triptolide, noted above, are not expected to undergo conversion to triptolide by a predictable mechanism, but nonetheless exhibit biological activities shown by triptolide (e.g. cytotoxicity in human T cell lymphoma (Jurkat) cells and immunosuppressive activity), as reported, for example, in the US applications and patents cited above. Compounds in this category are referred to herein as non-prodrug derivatives, or simply derivatives, of triptolide. (Note that while such compounds could in fact be converted to triptolide in vivo by a yet unknown mechanism, they are not designed for such conversion, as are triptolide prodrugs.) This category may also include prodrugs of triptolide derivatives, such as 14-acetyl-5,6-didehydro triptolide and 18-deoxo-19-dehydro-18-benzoyloxy-19-benzoyl triptolide. In one embodiment, the derivative is a synthetic derivative. Derivatives also include the naturally occurring compounds 16-hydroxytriptolide and tripdiolide (2-hydroxy triptolide).

Triptolide derivatives and prodrugs useful in the invention are not intended to be limited to the exemplary compounds discussed above.

With regard to structure, a “derivative” of triptolide preferably refers to a compound derived from triptolide via a modification which can include, for example: substitution of a hydrogen atom or hydroxyl group with hydroxyl, lower alkyl or alkenyl, lower acyl, lower alkoxy, lower alkyl amine, lower alkylthio, oxo (═O), or halogen; or conversion of a single bond to a double bond or to an epoxide. In this sense, “lower” preferably refers to C₁ to C₄; e.g. “lower alkyl” preferably refers to methyl, ethyl, or linear or branched propyl or butyl. Preferred hydrogen atom substitutions include hydroxyl, methyl, acetyl (C(O)CH₃) and fluoro.

Triptolide derivatives and prodrugs which are “immunosuppressive” can be identified via standard in vitro and in vivo assays. In vitro assays include the IL-2 inhibition assay described in co-owned PCT Pubn. No. WO 2003/101951. Compounds are preferably assayed for inhibition of TGF-β, using commercially available kits, such as the TGF-β Emax® ImmunoAssay System provided by Promega Corporation. Preferably, immunosuppressive activity is such that the target cytokine, such as IL-2 or, preferably, TGF-β, is inhibited by the triptolide derivative or prodrug at a concentration at most 500 times greater, more preferably at most 100 times greater, and most preferably at most 50 times greater than the concentration of native triptolide that provides the same level of inhibition in the same assay or system. In some embodiments, the triptolide derivative or prodrug is effective at a concentration of about 10-50 times the effective concentration of triptolide, or it may be effective at a concentration of about 1-10 times the effective concentration of triptolide in the same assay.

II. Therapeutic Compositions

Formulations containing triptolide compounds may take the form of solid, semi-solid, lyophilized powder, or liquid dosage forms, such as tablets, capsules, powders, sustained-release formulations, solutions, suspensions, emulsions, ointments, lotions, or aerosols, preferably in unit dosage forms suitable for simple administration of precise dosages. The compositions typically include a conventional pharmaceutical carrier or excipient and may additionally include other medicinal agents, carriers, or adjuvants.

Preferably, the composition will be about 0.5% to 75% by weight of a triptolide compound or compounds, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

The composition may be administered to a subject orally, transdermally or parenterally, e.g., by intravenous, subcutaneous, intraperitoneal, or intramuscular injection. For use in oral liquid preparation, the composition may be prepared as a solution, suspension, emulsion, or syrup, being supplied either in liquid form or a dried form suitable for hydration in water or normal saline. For parenteral administration, an injectable composition for parenteral administration will typically contain the triptolide derivative in a suitable intravenous solution, such as sterile physiological salt solution.

Liquid compositions can be prepared by dissolving or dispersing the triptolide compound (generally about 0.5% to about 20%) and optional pharmaceutical adjuvants in a pharmaceutically acceptable carrier, such as, for example, aqueous saline, aqueous dextrose, glycerol, or ethanol, to form a solution or suspension.

The compound may also be administered by inhalation, in the form of aerosol particles, either solid or liquid, preferably of respirable size. Such particles are sufficiently small to pass through the mouth and larynx upon inhalation and into the bronchi and alveoli of the lungs. In general, particles ranging from about 1 to 10 microns in size, and preferably less than about 5 microns in size, are respirable. Liquid compositions for inhalation comprise the active agent dispersed in an aqueous carrier, such as sterile pyrogen free saline solution or sterile pyrogen free water. If desired, the composition may be mixed with a propellant to assist in spraying the composition and forming an aerosol.

Methods for preparing such dosage forms are known or will be apparent to those skilled in the art; for example, see Remington's Pharmaceutical Sciences (20th Ed., Lippincott Williams & Wilkins., 2000). The composition to be administered will contain a quantity of the selected compound in an effective amount for effecting immunosuppression, particularly cytokine inhibition, in a patient afflicted with an inflammatory disorder as described herein.

III. Treatment Methods

In accordance with the invention, administration of a triptolide compound is expected to inhibit cytokine production, particularly TGF-β production, in a patient suffering from obliterative airway disease, renal fibrosis, diabetic nephropathy, or liver fibrosis, thereby reducing the symptoms of the disease. A range of doses is practical for this treatment. Results from a phase I clinical trial with a triptolide prodrug, triptolide succinate sodium salt (designated PG490-88Na), show that a dose of 0.675 μg/m² administered by i.v. infusion is well tolerated with no drug-related toxicity. This dose calculates to about 20 μg/kg. Treatment in this clinical study is administered at weekly intervals.

For administration to human patients, a reasonable range of doses, for a prodrug that converts to triptolide in human plasma at a rate similar to that of triptolide succinate, is 1-100 μg/kg. For derivatives which do not require conversion for activity, such as 14-deoxy-14α-fluoro triptolide, a lower dose range will be useful, such as 0.1 to 40 μg/kg, depending upon the activity of the derivative compared to that of triptolide.

In some cases, patients may be treated several times per day by i.v. infusion with the triptolide prodrugs or derivatives, or possibly by continuous infusion, as dictated by their clinical state and response to the treatment. With more frequent, or continuous treatment, the dose on a μg/m² or μg/kg basis would be reduced. While i.v. administration is preferred in a clinical setting, other modes of administration, such as parenteral or oral, may also be used, with higher dosages typically used for oral administration.

Liquid compositions can be prepared by dissolving or dispersing the triptolide compound (generally about 0.5% to about 20%) and optional pharmaceutical adjuvants in a pharmaceutically acceptable carrier, such as, for example, aqueous saline, aqueous dextrose, glycerol, or ethanol, to form a solution or suspension.

For use in oral liquid preparation, the composition may be prepared as a solution, suspension, emulsion, or syrup, being supplied either in liquid form or a dried form suitable for hydration in water or normal saline. For i.v. or parenteral administration, of which the latter includes subcutaneous, intraperitoneal, or intramuscular injection, an injectable composition will typically contain the triptolide derivative in a suitable intravenous solution, such as sterile physiological salt solution.

The compound may also be administered by inhalation, particularly for treatment of OAD, in the form of aerosol particles, either solid or liquid, preferably of respirable size. Such particles are sufficiently small to pass through the mouth and larynx upon inhalation and into the bronchi and alveoli of the lungs. In general, particles ranging from about 1 to 10 microns in size, and preferably less than about 5 microns in size, are respirable. Liquid compositions for inhalation comprise the active agent dispersed in an aqueous carrier, such as sterile pyrogen free saline solution or sterile pyrogen free water. If desired, the composition may be mixed with a propellant to assist in spraying the composition and forming an aerosol.

IV. Combination Treatment

The triptolide compounds may be used in combination with other agents. These additional agents include, but are not limited to, antiviral agents, corticosteroids, additional immunosuppressive agents, e.g. as described above, and immune potentiators.

Other compounds with immunosuppressive activity include, for example: azathioprine, brequinar, chlorambucil, 2-chloro deoxyadenosine, cyclosporin, cyclophosphamide, 15-deoxyspergualin, dexamethasone, everolimus, fluorouracil, leflunomide, mercaptopurine, methotrexate, mitomycin, mitoxantrone, mizoribine (bredinin), mycophenolate mofetil, prednisone, prednisolone, sirolimus (rapamycin), thalidomide, tacrolimus (FK506), thioguanine, and thiopurine).

The level of cytokines can also be reduced, and the morbidity and mortality of SARS reduced, by the use of biological agents that have specificity for any of the cytokines produced in a SARS infection or prevent binding of these cytokines to cytokine receptors on target cells. Cytokine antagonists comprised of soluble receptors, antibodies, or binding proteins for the cytokines, or receptors to the cytokines, produced in a SARS infection may contribute to reduction in the cytokine levels. Cytokines such as TNF-α, IL-1β, IL-6, IL-8, IL-18 and others may be involved in the pathogenesis of SARS, and cytokine antagonists that bind to these or other cytokines or their receptors may prevent their biological effects and thus reduce the morbidity and mortality of the SARS infection. Etanercept™ (a soluble TNF receptor antagonist), Infliximab™ (an anti-TNF antibody) and Anakinra™ (a soluble IL-1 receptor antagonist) are examples of cytokine antagonists, and reagents targeting these and other cytokines/cytokine receptors are in preclinical and clinical development.

More than one of the cytokine antagonists described herein may be used in combination. The cytokine antagonists are specifically targeted at a single cytokine pathway. Combination treatment with triptolide compounds, immunosuppressive agents, and cytokine antagonists may be used to increase the effectiveness of the treatment.

As in any immunosuppressive therapy, it is advisable to monitor aspects of the immune system, to allow modulation of the treatment if necessary.

### EXAMPLES

Methods of synthesis of various exemplary triptolide derivatives and prodrugs are provided below. All structures were verified by NMR.

### Example 1

### Preparation of 14-C-methyltriptolide (PG670)

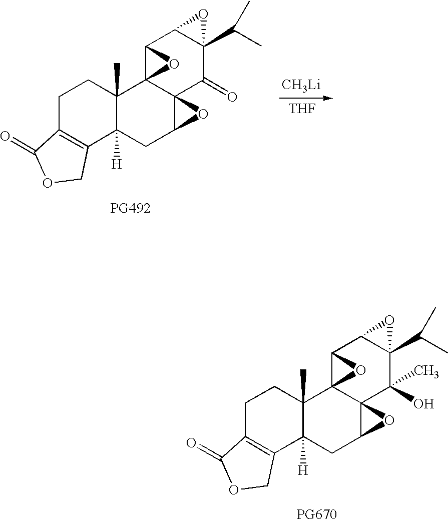

To a solution of triptonide (designated PG492) (60 mg, 0.17 mmol) in THF (5 ml) at −78° C. was added 0.45 ml of methyl lithium (1.4 M solution in ethyl ether, 0.63 mmol, 3.7 eq) under N₂. The solution was stirred at −78° C. for 2 hrs 45 mins and then at room temperature for 2 hrs, at which time the starting material had disappeared on TLC. Acetic acid (1 ml) was slowly added. The solution was then concentrated under vacuum. The crude product was dissolved in dichloromethane (3 ml) and passed through a pad of silica gel, which was then washed with 5% methanol in ethyl acetate (80 ml). After removal of solvent, 78 mg of crude product was obtained. This was dissolved in acetonitrile (0.6 ml) and filtered. The product mixture was separated on HPLC, using a 10×250 mm column of Econosil C18 and a guard column cartridge (7.5×4.6 mm) of Alltima C18, both from Alltech, with mobile phase CH₃CN/H₂O 40/60 with a flow rate of 2.0 ml/min. The sixth peak, having a retention time of 32.13 mins, was collected and concentrated under vacuum. The product had m/z 374 (7.9 mg, yield: 12.6%).

### Example 2

### Synthesis of Triptolide 14-tert-Butyl Carbonate (PG695)

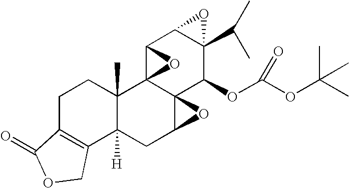

To a solution of triptolide (108.1 mg, 0.30 mmol, 1.0 eq) and 4-DMAP (367.0 mg, 3.0 mmol, 10.0 eq) in dichloromethane (15 ml) was added with stirring di-tert-butyl dicarbonate (393.0 mg 1.80 mmol, 6.0 eq) at room temperature under nitrogen. After 48 hours of stirring at room temperature, methyl alcohol (1.0 ml) was added. The reaction mixture was concentrated under vacuum and the crude product was purified via preparative TLC (EtOAc/hexanes/MeOH 1:1:0.1) to give 131.3 mg (95.1%) of product.

### Example 3

### Preparation of 14-deoxy-14α-fluoro triptolide

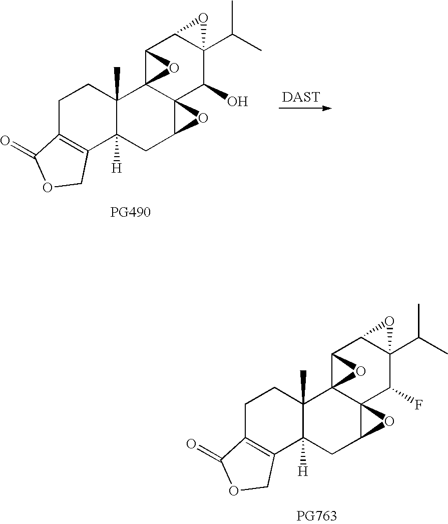

To a solution of PG490 (triptolide, 17.3 mg, 0.048 mmol) in dichloromethane (1.0 ml) at 0° C. was added (diethylamino)sulfur trifluoride (DAST, 100 μl, 0.763 mmol) under N₂. The reaction mixture was stirred at 0° C. for 2 hrs, and saturated NaHCO₃ solution (0.8 ml) was then added. The reaction mixture was extracted with 3×2 ml of dichloromethane. The combined organic layer was dried over anhydrous Na₂SO₄ and concentrated under vacuum. The product (PG763) was obtained in quantitative yield.

### Example 4

### Synthesis of 5-α-hydroxytriptolide (PG701)

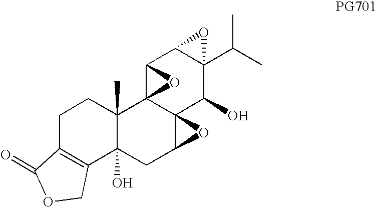

To a solution of triptolide (437.6 mg, 1.21 mmol) in 1,4-dioxane (35 mL) was added selenium dioxide (305.1 mg, 2.75 mmol). The reaction mixture was stirred at 90° C. under N₂ for 70 hrs. After cooling to room temperature, the reaction mixture was filtered through Celite and concentrated under vacuum. The crude product was purified via preparative TLC (EtOAc/CH₂Cl2 3:7) to yield the desired product (211.7 mg, 46.3%).

### Example 5

### Synthesis of 14-acetyl-5,6-didehydrotriptolide (PG746)

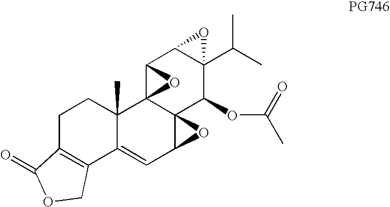

To a solution of 5-α-hydroxytriptolide (PG701, 98.3 mg, 0.261 mmol), 4-dimethylaminopyridine (DMAP, 45.2 mg) and triethylamine (TEA, 0.50 mL) in dichloromethane (5.0 mL) was added acetic anhydride (0.247 mL, 2.61 mmol, 10.0 eq) at room temperature under nitrogen. After stirring for 4-5 hrs at room temperature, methanol (1.0 mL) was added, and the reaction mixture was concentrated under vacuum. The crude product, 14-acetyl-5-α-hydroxytriptolide, was purified using preparative TLC. To a solution of this material (10.5 mg, 0.025 mmol), in CH₂Cl₂ (0.50 mL) at 0° C. was added (diethylamino)sulfur trifluoride (DAST, 4.3 μL, 0.033 mmol, 1.3 eq). The reaction mixture was stirred at 0° C. under N₂ for 40 mins. Saturated aq. NaHCO₃ (0.2 mL) diluted with 0.3 mL H₂O was added to the reaction mixture at 0° C. The mixture was then extracted with dichloromethane (1.5, 2×2.0 mL). The combined organic layers were dried over anhydrous Na₂SO₄ and concentrated under vacuum. The crude product was purified using preparative TLC (EtOAc/hexanes/MeOH 40:60:5.0) to yield 4.0 mg product (39.8%).

### Example 6

### Preparation of 19-Methyl Triptolide (PG795)

A. Protection of 14-hydroxyl group

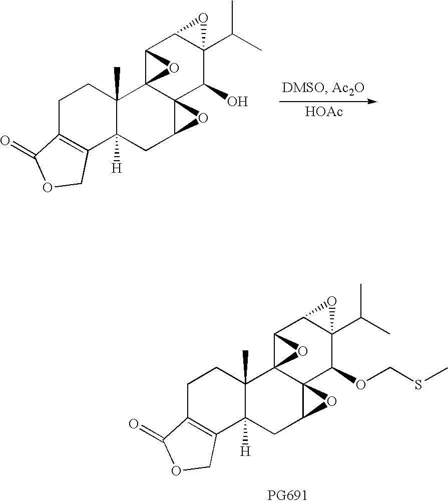

To a solution of triptolide (0.56 g, 1.6 mmol) in DMSO (8.5 mL, 0.12 mol) was added acetic acid (28 mL, 0.49 mol) and acetic anhydride (5.6 mL, 59 mol), and the solution was stirred at room temperature for five days. The reaction mixture was poured into 200 mL of water and neutralized with solid NaHCO₃, added in portions. The mixture was extracted with ethyl acetate (150 mL×3), and the extract was dried over anhydrous sodium sulfate and concentrated to give an oil. Silica gel column chromatography purification (3:2 hexanes/EtOAc) gave the intermediate (PG691) (0.45 g, 69%) as a white foam.

B. Methylation

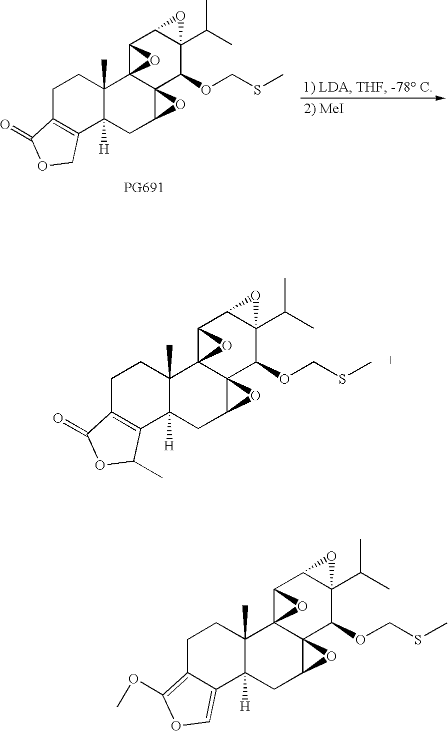

To a solution of PG691 (0.22 g, 0.52 mmol) in anhydrous TBF (10 mL) was added a solution of LDA in heptane/TBF/ethyl benzene (0.30 mL of 2.0 M solution, 0.60 mmol) dropwise at −78° C. The solution was stirred at this temperature for 15 min, followed by dropwise addition of CH₃I (50 μL, 0.80 mmol). The mixture was stirred at −78° C. for 2 h, then allowed to come to room temperature overnight.

The reaction mixture was neutralized with 1N HCl and extracted with EtOAc (10 mL×3). The EtOAc solution was washed with 5% aqueous sodium thiosulfate (10 mL×2) and dried over anhydrous sodium sulfate. Concentration under reduced pressure gave an oil. Column purification (silica gel, 3:2 hexanes/EtOAc) gave two products, methylthiomethyl protected 19-methyltriptolide (45.9 mg, 20%) and methylthiomethyl protected 18-methoxyfuranotriptolide.

C. Deprotection

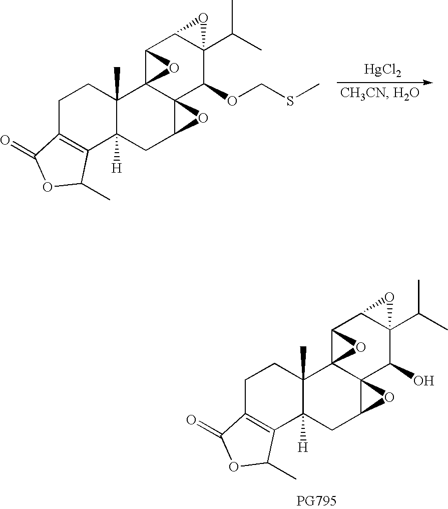

To a solution of methylthiomethyl protected 19-methyltriptolide, prepared as described above (45.9 mg, 0.106 mmol), in 1.5 mL acetonitrile/water (4:1) was added mercuric chloride (0.285 g, 1.05 mmol) in one portion. The resulting solution was stirred at room temperature overnight. The white solid which precipitated from the solution was removed by filtration through Celite® and rinsed with ethyl acetate. The EtOAc solution was washed twice with 5% aqueous NH₄OAc. The organic phase was dried (Na₂SO₄) and concentrated under reduced pressure to give the crude product. Purification by column chromatography (silica gel, 1:1 hexanes/ethyl acetate) gave the product (39.5 mg, 99%).

### Example 7

### Preparation of 18-deoxo-19-dehydro-18-benzoyloxy-19-benzoyl triptolide (PG796)

A. Acylation

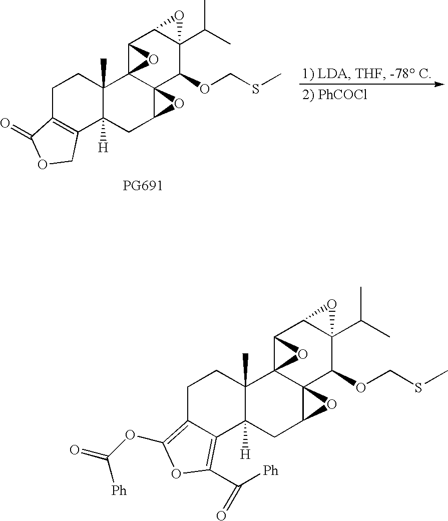

To a solution of PG691, prepared as described above (73.1 mg, 0.174 mmol), in anhydrous THF (5 mL) was added a solution of LDA in heptane/THF/ethyl benzene (0.34 mmol) dropwise at −78° C. The solution was stirred at this temperature for 15 min, followed by the dropwise addition of benzoyl chloride (100 μL, 0.86 mmol). The reaction was stirred at −78° C. for 2 h, then quenched with water and extracted with ethyl acetate (25 mL×3). The combined organic solution was dried over anhydrous sodium sulfate. Concentration under reduced pressure gave an oil. Column purification (silica gel, 3:2 hexanes/ethyl acetate) gave the 14-protected product (51.2 mg, 47%).

B. Deprotection

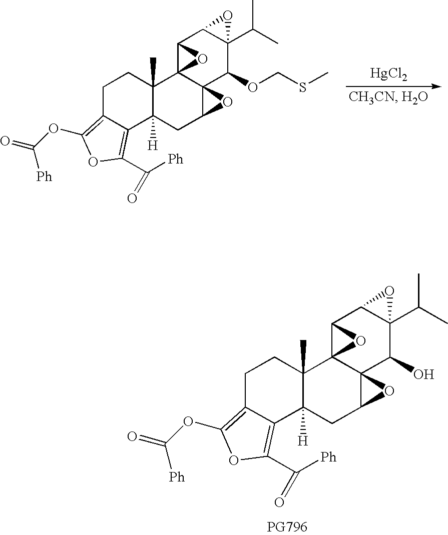

To a solution of the 14-methylthiomethyl protected product, prepared as described above (51.2 mg, 0.0814 mmol), in 1.5 mL acetonitrile/water (4:1) was added mercuric chloride (0.22 g, 0.81 mmol) in one portion. The resulting solution was stirred at room temperature overnight. The white solid which precipitated from the solution was removed by filtration through Celite® and rinsed with ethyl acetate. The EtOAc solution was washed twice with 5% aqueous NH₄OAc. The organic phase was dried (Na₂SO₄) and concentrated under reduced pressure to give the crude product. Purification by column chromatography provided the pure product (32.8 mg, 71%).

### Example 8

### Synthesis of Triptolide 14-Ethyl Carbamate (PG666)

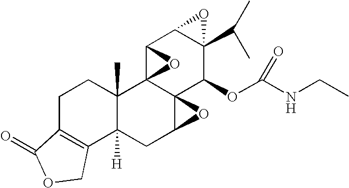

A mixture of triptolide (0.20 mmol, 1.0 eq) and ethyl isocyanate (3.0 mmol, 15.0 eq) in DMF (7.0 ml) was sealed and heated in a 54° C. oil bath with stirring. The reaction was monitored with TLC. After the starting material was completely consumed, the reaction mixture was concentrated under vacuum, and the crude product is purified with preparative TLC. The compound was obtained in 98.5% yield.

Analytical TLC Rf=0.44 (ethyl acetate/hexanes/methanol 1:1:0.1). IR (KBr): 3369.6, 2975.6, 2937.6, 2878.0, 1753.0, 1719.0, 1686.1, 1676.5, 1524.0, 1517.7, 1509.0, 1458.7, 1448.8, 1245.8, 1142.5, 1076.3, 1030.8, 988.1, 944.4, 866.9, 722.6, 560.5 cm⁻¹. H¹NMR (300 MHz, CDCl₃): δ=4.94 (1H, s, 14-CH), 4.68 (2H, s, 19-CH₂), 3.83 (1H, d, 11-CH), 3.51 (1H, d, 12-CH), 3.48 (1H, d, 7-CH), 3.26 {2H, m, 22-CH₂ (—NCH₂CH₃)}, 2.70 (1H, m, 5-CH), 2.32 (1H, m, 2-CHb), 2.13 (2H, m, 6-CHb and 2-CHa), 1.93 (2H, m, 15-CH and 6-CHa), 1.57 (1H, dd, 1-CHb), 1.22 (1H, m, 1-CHa), 1.16 {3H, t, 23-CH3 (—NCH₂CH₃)}, 1.07 (3H, s, 20-CH₃), 0.99 (3H, d, 17-CH₃), 0.86 (3H, d, 16-CH₃) ppm HRMS (FAB) m/z calcd for C₂₃H₃₀NO₇⁺ (MH⁺) 432.2022, found 432.2016.

### Example 9

### Synthesis of Triptolide Succinate (PG490-88)

Triptolide (100 mg) in 10 ml of pyridine was treated with succinic anhydride (150 mg) at room temperature. The reaction was carried out at 85° C. for 30 hours under a nitrogen atmosphere. Hexane (50 ml) was added to the resultant mixture to precipitate a crude product, which was collected by filtration and washed with hexane. The crude product was recrystallized from ether/hexane to yield 90 mg (70%) of triptolide succinate (YM-262), m.p. 111-113° C.

IR(KBr): 3431.8, 2974.6, 1743.8, 1375.5, 1159.4, 1022.4 cm⁻¹. H¹NMR(CDCl₃): 5.08 (1H, s, 14-CH), 4.67 (2H, s, 19-CH2), 3.82 (1H, d, 11-CH), 3.50 (1H, d, 12-CH), 3.43 (1H, d, 7-CH), 2.75 (5H, m, CH2CH2, 5-CH), 2.30(1H, d-m, 15-CH), 2.15 (2H, m 6-CHₐ, 2-CHₐ), 1.88 (2H, m, 2-CH_{b}, 6-CH_{b}B), 1.55 (1H, m, 1-CH_{b}), 1.20 (1H, m, −1-CHa), 1.05 (3H, s, 20-CH3), 0.95 (3H, d, 16-CH₃), 0.83 (3H, d, 17-CH₃) ppm. MS (m/z): 461 (M+1).

### Example 10

### Conversion of a Triptolide Prodrug to Triptolide in Blood Serum

A solution of triptolide succinate in DMSO (25 mg/ml, 0.1 ml) was mixed with 0.5 ml of rat serum. The mixture was incubated at 37° C. Aliquots of the mixture were taken at 1, 3, 5, 15, 45 minutes and 18 hours and analyzed by thin layer chromatography (TLC). The TLC plates were developed in 1:5 CH₂Cl₂/Et₂O. After development, the plates were treated with iodine vapor and examined under a UV lamp. Triptolide and triptolide succinate were used as reference compounds (R_{f}=0.60 and 0.45, respectively). After 3 minutes, only triptolide succinate was detected by TLC. After 15 minutes, the triptolide succinate spot had disappeared (R_{f}=0.45), and a new spot corresponding to triptolide had appeared (R_{f}=0.60).

### Example 11

### Cytokine Inhibition Assays

Test samples are diluted to 1 mM in complete tissue culture medium. Aliquots are placed in microculture plates coated with anti-CD3 antibody (used to stimulate the production of IL-2 by Jurkat cells), and serial dilutions are prepared so that the final concentrations encompass the range of 0.001 to 10,000 nM in log increments. Cells from an exponentially expanding culture of Jurkat human T cell line (e.g. #TIB-152 obtained from American Type Culture Collection, Manassas, Va.) are harvested, washed once by centrifugation, re-suspended in complete tissue culture medium, and diluted to a concentration of 2×10⁶ cells/ml. A volume of 50 μl of Jurkat cells (1×10⁵ cells) is added to wells containing 100 μl of the diluted compounds, 50 μl of PMA (10 ng/ml) is added to each well, and the plates are incubated at 37° C. in a 5% CO₂ incubator. After 24 hours, the plates are centrifuged to pellet the cells, 150 μl of supernatant is removed from each well, and the samples are stored at −20° C. The stored supernatants can be analyzed for human IL-2 concentration using the Luminex 100 (Luminex Corporation, Austin, Tex.), Luminex microspheres coupled with anti-IL-2 capture antibody, and fluorochrome-coupled anti-IL-2 detection antibody. The data are expressed and preferably plotted as concentration of compound versus IL-2 concentration (pg/ml).

Alternatively, TGF-β levels can be assayed in control and compound-treated cell cultures, using a commercially available TGF-β ELISA kit (TGF-β E max ImmunoAssay System; Promega Corp., Madison, Wis.). The kit contains a TGF-β coat monoclonal antibody for a 96-well microtiter plate coating and immunomobilized mouse polyclonal antibody to TGF-β with a reported sensitivity of 15.6 μg/ml. Sample is acidified to convert TGF-β from a latent form to the immunoreactive form detected by the anti-TGB-β antibody. The representative standard curve is generated using the TGF-β standard provided with the kit.

1. A method of inhibiting cytokine production in a patient infected with an inflammatory disorder selected from obliterative airway disease, renal fibrosis, diabetic nephropathy, and liver fibrosis, thereby reducing symptoms of the disorder, comprising administering to such a patient an immunosuppressive triptolide compound.

2. The method of claim 1, wherein the cytokine is TGF-β.

3. The method of claim 2, wherein the compound is triptolide.

4. The method of claim 2, wherein the compound is a triptolide prodrug.

5. The method of claim 2, wherein the triptolide compound is an immunosuppressive derivative of triptolide or a prodrug thereof.

6. The method of claim 4, wherein said compound is selected from the group consisting of triptolide 14-succinate, triptolide 14-t-butyl carbonate, triptolide 14-ethylcarbamate, and triptolide 14-(α-dimethylamino)acetate.

7. The method of claim 5, wherein said compound is selected from the group consisting of 14-methyl triptolide, triptolide 14-tert-butyl carbonate, 14-deoxy-14α-fluoro triptolide, triptolide 14-(α-dimethylamino)acetate, 5-α-hydroxy triptolide, 14-acetyl-5,6-didehydro triptolide, 19-methyl triptolide, and 18-deoxo-19-dehydro-18-benzoyloxy-19-benzoyl triptolide.

8. The method of claim 1, wherein the triptolide compound is employed in combination with an additional therapeutic agent selected from an antiviral agent, an antiinflammatory agent, an additional immunosuppressive agent, and an immune potentiator.

9. The method of claim 1, wherein said disorder is obliterative airway disease.

10. The method of claim 1, wherein said disorder is renal fibrosis.

11. The method of claim 1, wherein said disorder is diabetic nephropathy.

12. The method of claim 1, wherein said disorder is liver fibrosis.
